(19) United States
(12) Patent Application Publication
(10) Pub. No.: US 2012/0035361 A1
(43) Pub. Date: Feb. 9, 2012

(54) SELECTIVE INHIBITORS OF EXCITATORY AMINO ACID TRANSPORTER SUBTYPE 1 (EAAT1/GLAST)

(76) Inventors: Lennart Bunch, Copenhagen S (DK); Anders Asbjørn Jensen, Copenhagen S (DK)

(21) Appl. No.: 13/157,353

(22) Filed: Sep. 19, 2011

(30) Foreign Application Priority Data
Dec. 12, 2008 (DK) PA/2008/01768

(51) Int. Cl. C07D 311/74; C07D 413/12; C07D 405/04; C07D 409/04

(52) U.S. Cl. 544/151; 549/404; 549/60; 546/282.7; 548/464

(57) ABSTRACT

The invention is the discovery of the use of the class of compounds represented by Formula I, as selective inhibitors of excitatory amino acid transporter (EAAT) subtype 1 (EAAT1) and its rodent ortholog L-glutamate/L-aspartate transporter (GLAST) for the study of function and distribution of EAAT1/GLAST in the central nervous system and studies of the physiological and pathological functions of the EAAT1/GLAST subtype in native tissues, cultured neurons, and/or animal models for CNS disorders.

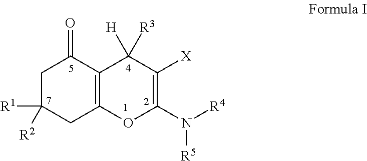

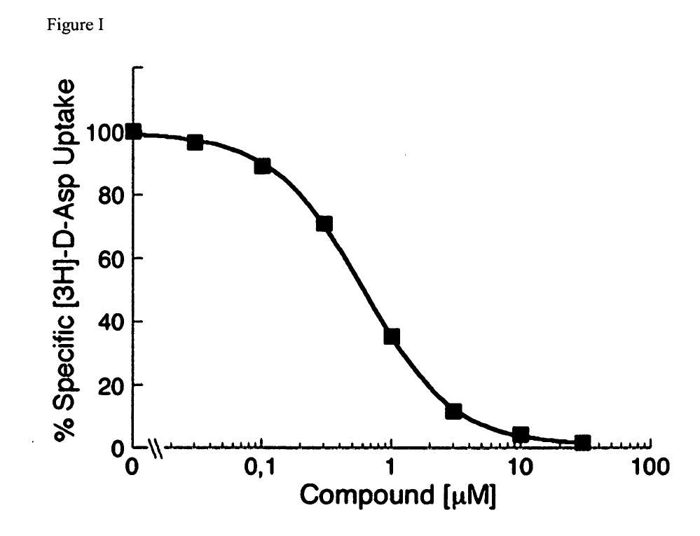

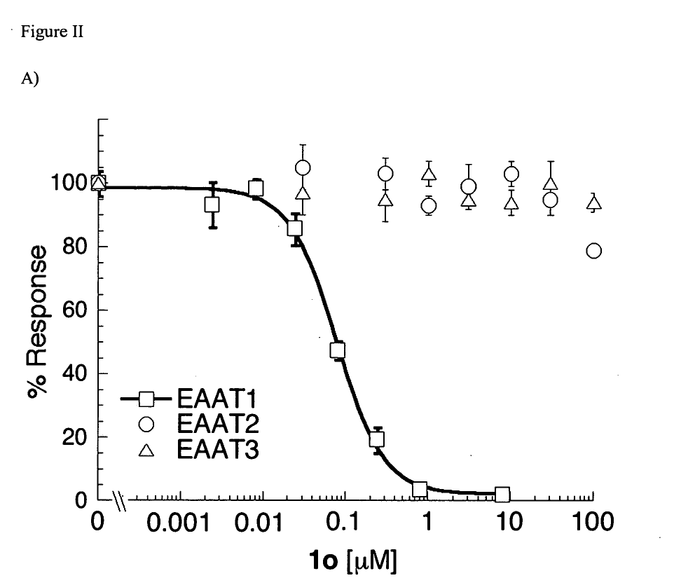

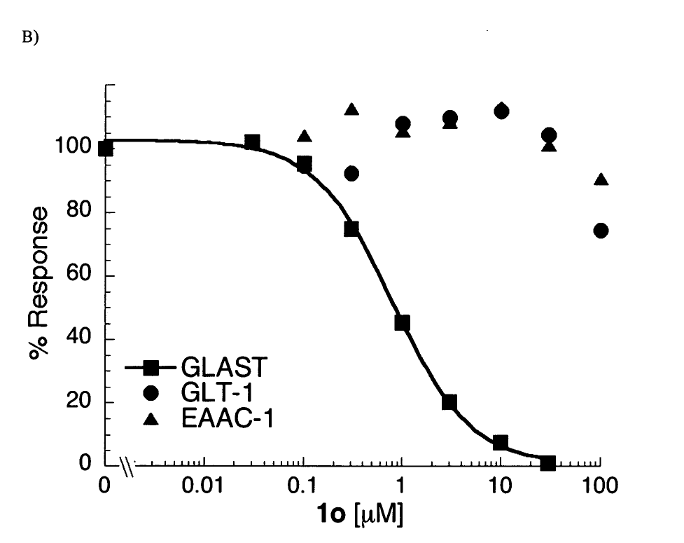

### CROSS-REFERENCE TO RELATED APPLICATIONS

Priority is claimed to the following patent applications:

1) PCT application (WO/2010/066253) “SELECTIVE INHIBITORS OF EXCITATORY AMINO ACID TRANSPORTER SUBTYPE 1 (EAAT1/GLAST)”, by Lennart Bunch and Anders Asbjørn Jensen, submitted Dec. 09, 2009

2) National, Denmark: (PA/2008/01768) “SELECTIVE INHIBITORS OF HUMAN EXCITATORY AMINO ACID TRANSPORTER SUBTYPE 1”, by Lennart Bunch and Anders Asbjørn Jensen, submitted: Dec. 12, 2008

### BACKGROUND OF THE INVENTION

This invention is within the field of medicinal chemistry. In details, the invention covers the use of substituted 2-amino-5-oxo-5,6,7,8-tetrahydro-4H-chromene-3-carbonitrile as the first selective inhibitors of excitatory amino acid transporter subtype 1 (EAAT1/GLAST) to study the function of this transporter subtype (EAAT1/GLAST). Since this is the first selective inhibitor of EAAT1/GLAST it represents a novel pharmacological tool.

Two patent applications describe the use of structurally related analogs for other purposes:

PCT application WO 00/75123A1 broadly describes the potential use of a series of compounds structurally related to the compound series covered by this application, as AMPA receptor potentiators. However, the specific compounds synthesized and pharmacologically characterized in WO 00/75123A1 are not a part of this patent application and we have shown that they display no activity at EAAT1/GLAST. Furthermore, on the basis of molecular structure and function of AMPA receptors and EAATs there is no scientific basis justifying the likeliness for cross activity of a compound at these two targets.

Patent application US 2007/0148185A1 describes specifically the compound (4S,7R)-2-amino-4-isobutyl-5-oxo-7-phenyl-5,6,7,8-tetrahydro-4H-chromene-3-carbonitrile and its potential use as an anti-parasitic drug.

### SUMMARY OF THE INVENTION

Structurally defined compounds according to claims 1-4 represent the first class of selective EAAT1/GLAST inhibitors. Such compounds will enable the investigation of the function and distribution of EAAT1/GLAST in the central nervous system, and in studies of the physiological and pathological functions of the EAAT1/GLAST subtype in native tissues, cultured neurons, and/or animal models for CNS disorders (claim 5).

### DESCRIPTION OF DRAWINGS

FIG. I: Concentration-inhibition curve for compound 1o at EAAT1.

FIG. II: Concentration-inhibition curves for compound 1o at human EAAT subtypes EAAT1, EAAT2 and EAAT3 stably expressed in HEK293 cell lines (A) and at the rat EAAT subtypes GLAST, GLT-1 and EAAC-1 transiently expressed in tsA201 cells (B) in the FLIPR® Membrane Potential (FMP) Blue assay.

Table 1: Inhibition of [³H]-D-Asp uptake in HEK293 cells stably expressing human EAAT1, EAAT2 and EAAT3. The IC₅₀ values are given in μM with pIC₅₀±S.E.M. values in brackets (n=3-7).

### DETAILED DESCRIPTION OF THE INVENTION

The invention is the class compounds with the general structure depicted in Formula I are selective inhibitors of human excitatory amino acid transporter (EAAT) subtype 1 (EAAT1) and its corresponding rodent ortholog L-glutamate/L-aspartate transporter (GLAST).

R¹ is aryl, fused aryl, a carbocyclic group, a heterocyclic group, a heteroaryl group, alkenyl, alkynyl, arylalkyl, arylalkenyl, arylalkynyl, heteroarylalkyl, heteroarylalkenyl, heteroarylalkynyl, cycloalkylalkyl, heterocycloalkyl; or R¹ and R² are taken together to form a carbocycle or heterocycle; and

R² is hydrogen, C₁₋₁₀ alkyl, haloalkyl, aryl, fused aryl, a carbocyclic group, a heterocyclic group, a heteroaryl group, alkenyl, alkynyl, arylalkyl, arylalkenyl, arylalkynyl, heteroarylalkyl, heteroarylalkenyl, heteroarylalkynyl, cycloalkylalkyl, heterocycloalkyl, hydroxyalkyl, amino alkyl or thioalkyl; and

R³ is C₁₋₁₀ alkyl, haloalkyl, aryl, fused aryl, a carbocyclic group, a heterocyclic group, a heteroaryl group, alkenyl, alkynyl, arylalkyl, arylalkenyl, arylalkynyl, heteroarylalkyl, heteroarylalkenyl, heteroarylalkynyl, cycloalkylalkyl, heterocycloalkyl, hydroxyalkyl, amino alkyl or thioalkyl; and

R⁴ and R⁵ are independently hydrogen, C₁₋₁₀ alkyl, haloalkyl, aryl, fused aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl or aminoalkyl; or R⁴ and R⁵ are taken together to form a heterocycle; and

X is halogen, alkyl, aryl, heteroaryl, —NO₂ or —CN;

Any radiolabled analog as well as any pharmaceutically acceptable salt or prodrug thereof.

For Formula I, in where R¹⁻⁵ are independently a phenyl group which comprises the substituents, R⁶⁻¹⁰ (Formula II),

R⁶⁻¹⁰ are independently hydrogen, C₁₋₁₀ alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, nitro, amino, cyano, acylamino, hydroxy, thiol, acyloxy, azido, alkoxy, carboxy, carbonylamido, alkylthiol, halogen, haloalkyl, aryl, aryloxy, arylthioxy fused aryl, a carbocyclic group, a heterocyclic group, a heteroaryl group, C₁₋₀ alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, nitro, amino, cyano, acylamido, hydroxy, thiol, acyloxy, azido, alkoxy, carboxy, carbonylamido or alkylthiol; or

R⁶ and R⁷, or R⁷ and R⁸, or R⁸ and R⁹, or R⁹ and R¹⁰ , are taken together to form a carbocycle or heterocycle, including —OCH₂O—, —OCF₂O—, —(CH₂)₃—, —(CH₂)₄—, —OCH₂CH₂O—, —CH₂N(R)CH₂—, —CH₂CH₂N(R)CH₂—, —CH₂N(R)CH₂CH₂—, —CH═CH—CH═CH—, and —N=CH—CH=N—.

With respect to the invention disclosed here, the following definitions apply, unless otherwise noted:

Useful aryl groups are C₆₋₁₄ aryl, especially C₆₋₁₀ aryl. Typical C₆₋₁₄ aryl groups include phenyl, naphthyl, phenanthryl, anthracyl, indenyl, azulenyl, biphenyl, biphenylenyl and fluorenyl groups.

Useful cycloalkyl groups are C₃₋₈ cycloalkyl. Typical cycloalkyl groups include cyclopropyl, cyclobutyl, cyclopentyl, cyclohexyl and cycloheptyl. The term “heteroaryl” as employed herein refers to groups having 5 to 14 ring atoms; 6, 10 or 14 it electrons shared in a cyclic array; and containing carbon atoms and 1, 2, 3 or 4 oxygen, nitrogen or sulfur heteroatoms (where examples of heteroaryl groups are: thienyl, benzo[b]thienyl, naphtho[2,3b]thienyl, thianthrenyl, furyl, benzofuryl, pyranyl, isobenzofuranyl, benzoxazonyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, peridinyl, 4aH-carbazolyl, carbazolyl, β-carbolinyl, phenanthridinyl, acridinyl, perimidinyl, phenanthrolinyl, phenazinyl, thiazolyl, isothiazolyl, phenothiazinyl, isoxazolyl, furazanyl, tetrazolyl, triazolyl, and phenoxazinyl groups).

The terms “halo” or “halogen” refer to fluorine, chlorine, bromine and iodine atoms.

The term “alkyl groups” refers to straight-chained and branched C₁₋₁₀ alkyl groups. Typical C₁₋₁₀ alkyl groups include methyl, ethyl, propyl, isopropyl, butyl, sec-butyl, tert-butyl, iso-butyl, 3-pentyl, hexyl, heptyl, and octyl groups.

The term “alkenyl groups” refers to C₂₋₆ alkenyl groups, preferably C₂₋₄ alkenyl. Typical C₂₋₄ alkenyl groups include ethenyl, propenyl, isopropenyl, butenyl, and sec-butenyl.

The term “alkynyl groups” refers to C₂₋₆ alkynyl groups, preferably C₂₋₄ alkynyl. Typical C₂₋₄ alkynyl groups include ethynyl, propynyl, butynyl, and 2-butynyl groups.

The terms “arylalkyl” or “aralkyl groups” refer to any of the above-mentioned C₁₋₁₀ alkyl groups substituted by any of the above-mentioned C₆₋₁₄ aryl groups. The term “arylalkenyl groups” refers to any of the above-mentioned C₂₋₄ alkenyl groups substituted by any of the above-mentioned C₆₋₁₄ aryl groups.

The term “arylalkynyl groups” refers to any of the above-mentioned C₂₋₄ alkynyl groups substituted by any of the above-mentioned C₆₋₁₄ aryl groups.

The term “heteroarylalkyl groups” refers to any of the above-mentioned C₁₋₁₀ alkyl groups substituted by any of the above-mentioned heteroaryl groups.

The term “heteroarylalkenyl groups” refers to any of the above-mentioned C₂₋₄ alkenyl groups substituted by any of the above-mentioned heteroaryl groups.

The term “heteroarylalkynyl groups” refers to any of the above-mentioned C₂₋₄ alkynyl groups substituted by any of the above-mentioned heteroaryl groups. The term “cycloalkylalkyl groups” refers to any of the above-mentioned C₁₋₁₀ alkyl groups substituted by any of the above-mentioned cycloalkyl groups.

The term “haloalkyl groups” refers to C₁₋₁₀ alkyl groups substituted by one or more fluorine, chlorine, bromine or iodine atoms, e.g. fluoromethyl, difluoromethyl, trifluoromethyl, pentafluoroethyl, 1, 1-difluoroethyl and trichloromethyl groups.

The term “ydroxyalkyl groups” refers to C₁₋₁₀ alkyl groups substituted by hydroxy, e.g. hydroxymethyl, hydroxyethyl, hydroxypropyl and hydroxybutyl groups.

The term “alkoxy groups” refers to oxygen substituted by one of the C₁₋₁₀ alkyl groups mentioned above.

The term “alkylthio groups” refers to sulfur substituted by one of the C₁₋₁₀ alkyl groups mentioned above.

The term “acylamino groups” refers to any C₁₋₆ acyl (alkanoyl) attached to an amino nitrogen, e.g. acetamido, propionamido, butanoylamido, pentanoylamido, hexanoylamido as well as aryl-substituted C₂₋₆ substituted acyl groups.

The term “acyloxy groups” refers to any C₁₋₆ acyl (alkanoyl) attached to an oxy (—O—) group, e.g. acetoxy, propionoyloxy, butanoyloxy, pentanoyloxy, hexanoyloxy and the like.

The term “heterocycle” refers to saturated or partially unsaturated 3-7 membered monocyclic, or 7-10 membered bicyclic ring system, which consists of carbon atoms and from one to four heteroatoms independently selected from the group consisting of O, N, and S, wherein the nitrogen and sulfur heteroatoms can be optionally oxidized, the nitrogen can be optionally quaternized, and including any bicyclic group in which any of the above-defined heterocyclic rings is fused to a benzene ring, and wherein the heterocyclic ring can be substituted on carbon or on a nitrogen atom if the resulting compound is stable. Examples include, but are not limited to, pyrrolidine, piperazine, morpholine, imidazoline, pyrazolidine, benzodiazepines and the like.

The term “heterocycloalkyl groups” refers to any of the above-mentioned C₁₋₁₀ alkyl groups substituted by any of the above-mentioned heterocyclic groups.

The terms “alkylamino” and “dialkylamino groups” are —NHR₂₀ and —NR₂₀R₂₁, wherein R₂₀ and R₂₁ are C₁₋₁₀ alkyl groups.

Aminocarbonyl group is -C(O)NH₂.

The term “alkylaminocarbonyl groups” are carbonyl groups substituted by NHR₂₀ or —NR₂₀R₂₁, wherein R₂₀ and R₂₁ are C₁₋₁₀ alkyl groups.

The term “alkylthiol groups” includes any of the above-mentioned C₁₋₁₀ alkyl groups substituted by a —SH group.

A carbamoyloxy group is —O—C(O) —NH₂.

A carboxy group is —COOH.

An azido group is —N₃.

An ureido group is —NH—C(O)—NH₂.

An amino group is —NH₂.

An amide group is —NHC(O)—.

Certain of the compounds of the present invention may exist as optical isomers and the invention includes both the racemic mixtures of such optical isomers as well as the individual enantiomers that may be separated according to methods that are well known to those ordinary skilled in the art.

Examples of pharmaceutically acceptable addition salts include inorganic and organic acid addition salts such as hydrochloride, hydrobromide, phosphate, sulphate, citrate, lactate, tartrate, maleate, fumarate, mandelate and oxalate.

Examples of prodrugs include esters or amides of Formula I with R¹⁻¹³ as hydroxyalkyl or aminoalkyl, by reacting such compounds with an anhydride such as succinic anhydride.

The invention disclosed herein is meant to encompass all pharmaceutically acceptable salts thereof of the disclosed compounds. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like.

The invention disclosed herein is also meant to encompass prodrugs of the disclosed compounds. Prodrugs are considered to be any covalently bonded carriers which release the active parent drug in vivo. Examples of prodrugs include esters or amides of Formula I with R¹⁻¹³ as hydroxyalkyl or aminoalkyl, and these may be prepared by reacting such compounds with anhydrides such as succinic anhydride.

The invention disclosed herein is also meant to encompass the in vivo metabolic products of the disclosed compounds. Such products may result for example from the oxidation, reduction, hydrolysis, amidation, esterification and the like of the administered compound, primarily due to enzymatic processes. Accordingly, the invention includes compounds produced by a process comprising contacting a compound of this invention with a mammal for a period of time sufficient to yield a metabolic product thereof. Such products typically are identified by preparing a radiolabelled compound of the invention, administering it parenterally in a detectable dose to an animal such as rat, mouse, guinea pig, monkey, or to man, allowing sufficient time for metabolism to occur and isolating its conversion products from the urine, blood or other biological samples.

The invention disclosed herein is also meant to encompass the disclosed compounds being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, sulfurous, fluorine, chlorine, and iodine such as ²H, ³H, ¹³C, ¹⁴C, ¹⁵N, ¹⁷O, ¹⁸O, ¹⁸F, ³¹P, ³²P, ³⁵S, ³⁶Cl, and ¹²⁵I, respectively.

The compounds described by the general structure Formula I may contain one or more asymmetric centers and may thus give rise to enantiomers, diastereomers, and other stereoisomeric forms. The present invention is also meant to encompass racemic mixtures, resolved forms mixtures thereof, as well as the individual enantiomers that may be separated according to methods that are well know to those of ordinary skill in the art. When the compounds described herein contain olefinic double bonds or other centers of geometric asymmetry, and unless specified otherwise, it is intended to include both E and Z geometric isomers. All tautomers are intended to be encompassed by the present invention as well.

The term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The terms “enantiomer” or “enantiomeric” refer to a molecule that is not superimposable onto its mirror image and hence optically active wherein the enantiomer rotates the plane of polarized light in one direction and its mirror image rotates the plane of polarized light in the opposite direction.

The term “racemic” refers to a mixture of equal parts of enantiomers and which is optically inactive.

The term “resolution” refers to the separation or concentration or depletion of one of the two enantiomeric forms of a molecule. The phrase “enantiomeric excess” refers to a mixture wherein one enantiomer is present is a greater concentration than its mirror image molecule.

The invention is also directed to radiolabeled compounds of Formula I, in particular isotopes ³H, ¹⁴C, ¹⁸F, ¹²⁵I, and their use as radioligands for studying the EAAT1 subtype. Another use of the labeled compounds of the invention is an alternative to animal testing for the evaluation of structure-activity relationships.

Tritiated compounds of Formula I can be prepared by introducing tritium into the compound of Formula I by, for example, catalytic dehalogenation with tritium. This method includes reacting a suitably halogensubstituted precursor of a compound of Formula I with tritium gas in the presence of a suitable catalyst, for example Pd/C, in the presence or absence of a base. Other suitable methods for preparing tritiated compounds can be found in Filer, Isotopes in the Physical and Biomedical Sciences, Vol. 1, Labeled Compounds (Part A), Chapter 6. ¹⁴C-labeled compounds can be prepared by employing starting materials having a ¹⁴C carbon.

Also included within the scope of the present invention are the nontoxic pharmaceutically acceptable salts of the compounds of the present invention. Acid addition salts are formed by mixing a solution of the particular heteroaryl compound of the present invention with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, and the like. Basic salts are formed by mixing a solution of the heteroaryl compound of the present invention with a solution of a pharmaceutically acceptable non-toxic base such as sodium hydroxide, potassium hydroxide, choline hydroxide, sodium carbonate and the like.

The pharmaceutical compositions of the invention may be administered to any animal that may experience the beneficial effects of the compounds of the invention. Foremost among such animals are mammals, e.g., humans, although the invention is not intended to be so limited.

Compound Synthesis

The compounds of this invention, depicted by the general structure Formula I, may be prepared by methodology well known to those skilled in the art (multicomponent reaction). Various reaction conditions may apply: EtOH/amine base,[1] H₂O/catalytic HTMAB,[2] H₂O/DAHP,[3] and solvent-free/NaBr under microware conditions.[4] Given the chemical nature of the functional group X the compound series may be prepared (Scheme 1).

The appropriately 5-substituted cyclohexadienone A, aldehyde B, and C for use in the shown synthetic pathway are readily prepared according to literature procedures or commercially available.

The functional properties of the novel compounds of the invention were assessed in a [³H]-D-Aspartic acid uptake assay using HEK293 cell lines stably expressing the human EAAT subtypes EAAT1, EAAT2 and EAAT3. Furthermore, some of the compounds were assayed at human EAAT1, EAAT2 and EAAT3 and the rat orthologs GLAST, GLT-1 and EAAC-1 in the FLIPR® Membrane Potential (FMP) Blue assay (Molecular Devices, Crawley, UK).

FIG. I depicts a graph showing the concentration-inhibition curves for 1o at the human EAAT subtype EAAT1 stably expressed in HEK293 cell lines in a [³H]-D-aspartic acid uptake assay. The cells (grown in wells of 96-well plates) were washed twice with assay buffer, and incubated with buffer supplemented with 30 nM [³H]-D-aspartic acid in the absence (control) or presence of increasing concentrations of 1o at 3720 C. for 7 min. The assay mixtures were then quickly removed from the wells, and the cells were washed with 3 times with ice-cold buffer, scintillation fluid was added to each well, and the plate was counted in a TopCounter.

FIG. II depicts a graph showing the concentration-inhibition curves for 1o at human EAAT subtypes EAAT1, EAAT2 and EAAT3 stably expressed in HEK293 cell lines (A) and at the rat EAAT subtypes GLAST, GLT-1 and EAAC-1 transiently expressed in tsA201 cells (B) in the FLIPR® Membrane Potential (FMP) Blue assay (Molecular Devices, Crawley, UK). The cells (grown in wells of black clear bottom 96-well plates) were washed once with assay buffer and incubated with buffer supplemented with FMP assay dye (1 mg/ml) in the absence (control) or presence of increasing concentrations of to at 37° C. for 30 min. The plate was assayed at room temperature in a NOVOstar™ plate reader (BMG Labtechnologies, Offenburg, Germany) measuring emission at 560 nm caused by excitation at 530 nm before and up to 1 min after addition of an assay concentration of (S)-glutamic acid of 50 μM.

The synthesis and pharmacological evaluation of 1w (UCPH-102) is added to this patent application on Dec. 9, 2009. In comparison with 1o, compound 1w displays equipotency as a selective EAAT1 inhibitor over EAAT2,3 and is significantly more soluble in aq buffer solutions (pH=7.4), DMSO and now also CDCl₃.

Compositions within the scope of this invention include all compositions wherein the compounds of the present invention are contained in an amount which is effective to achieve its intended purpose. While individual needs vary, determination of optimal ranges of effective amounts of each component is with the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for psychosis disorders. Preferably, about 0.01 to about 10 mg/kg is orally administered to treat or prevent such disorders. For intramuscular injection, the dose is generally about one-half of the oral dose.

The pharmaceutical compositions of the invention may comprise the compounds of the present invention at a unit dose level of about 0.01 to about 50 mg/kg of body weight, or an equivalent amount of the pharmaceutically acceptable salt thereof, on a regimen of 1-4 times per day. Of course, it is understood that the exact treatment level will depend upon the case history of the animal or human, being treated. The precise treatment level can be determined by one of ordinary skill in the art without undue experimentation.

The unit oral dose may comprise from about 0.01 to about 50 mg, preferably about 0.1 to about 10 mg of the compound. The unit dose may be administered one or more times daily as one or more tablets each containing from about 0.1 to about 10, conveniently about 0.25 to 50 mg of the compound or its solvates.

In addition to, administering the compound as a raw chemical, the compounds of the invention may be administered as part of a pharmaceutical preparation containing suitable pharmaceutically acceptable earners comprising excipients and auxiliaries which facilitate processing of the compounds into preparations which can be used pharmaceutically. Preferably, the preparations, particularly those preparations which can be administered orally and which can be used for the preferred type of administration, such as tablets, dragees, and capsules, and also preparations which can be administered rectally, such as suppositories, as well as suitable solutions for administration by injection or orally, contain from about 0.01 to 99 percent, preferably from about 0.25 to 75 percent of active compound(s), together with the excipient.

Also included within the scope of the present invention are the nontoxic pharmaceutically acceptable salts of the compounds of the present invention. Acid addition salts are formed by mixing a solution of the particular compound (Formula I) of the present invention with a solution of a pharmaceutically acceptable non-toxic acid such as hydrochloric acid, fumaric acid, maleic acid, succinic acid, acetic acid, dichloroacetic acid, citric acid, tartaric acid, carbonic acid, phosphoric acid, oxalic acid, and the like. Basic salts are formed by mixing a solution of the particular compound

(Formula I) of the present invention with a solution of a pharmaceutically acceptable non-toxic base such as sodium hydroxide, potassium hydroxide, choline hydroxide, sodium carbonate and the like.

The pharmaceutical compositions of the invention may be administered to any animal which may experience the beneficial effects of the compounds of the invention. Foremost among such animals are mammals, e.g., humans, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as cetylcellulose phthalate or hydroxypropymethyl-cellulose phthalate, are used. Dye stuffs or pigment may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions may contain substances which increase the viscosity of the suspension include, sodium carboxymethyl cellulose, sorbitol, and/or dextran. Optionally, the suspension may also contain stabilizers.

The compounds of the present invention display pronounced EAAT1/GLAST-selectivity over the EAAT subtypes EAAT2/GLT-1 and EAAT3/EAAC-1, and thus they may be used to characterize this EAAT subtype and its distribution in the central nervous system. The compounds of the present invention which may be used for this purpose are isotopically radiolabelled derivatives, e.g. where one or more of the atoms are replaced with ³H, ¹¹C, ¹⁴C, ¹⁸F or ¹²⁵I. Alternatively, a fluorescent group may be employed. Examples of such groups include 4-nitrobenzofurazan and coumarines. Furthermore, the compounds of the present invention may be used as pharmacological tools in studies of the physiological and patophysiological functions of the EAAT1/GLAST subtype in native tissues.

Compound Synthesis

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

Chemistry. All reagents were obtained from commercial suppliers and used without further purification. The microwave reactions were carried out in a Biotage Initiator with autosampler (0-300 W at 2.45 GHz). Flash chromatography (FC) was performed using CombiFlash Companion System (Teledyne Isco, Inc.) on RediSep columns with silica gel (average particle size 35-70 microns). Vacuum liquid chromatography (VLC) was performed using silica gel (average particle size 15-40 microns). NMR (300 MHz) spectra were recorded in DMSO-D6 using DMSO as reference, unless otherwise noted. Melting points are uncorrected. 2-Amino-4-(4-methoxyphenyl)-7-(phenyl)-5-oxo-5,6,7,8-tetrahydro-4H-chromene-3-carbonitrile (1c). Equimolar amounts of 5-phenylcyclohexane-1,3-dione (0.095 g, 0.5 mmol), 4-methoxybenzaldehyde (61 μL, 0.5 mmol) and malononitrile (0.034 g, 0.5 mmol) were mixed together in H₂O/EtOH (1:1, 2 mL), and were allowed to react under microwave irradiation (reaction time: 30 min., temp.: 85° C., pre-stirring: 3 min., vial size: 2-5 mL, absorption level: high, fixed hold time: on). The reaction mixture was allowed to cool to rt, the solvent was filtered off and the solid obtained was washed with H₂O. Yield: 0.156 g (84%) white crystalline powder. Mp: 194.4-206.7° C. (decompose). ¹H NMR ε: 7.38-7.18 (m, 5H), 7.11 (d, 1H), 7.03-6.93 (m, 3H), 6.84 (d, 1H), 6.78 (d, 1H), 4.17 (s, 0.5H), 4.16 (s, 0.5H), 3.71 (s, 1.5H), 3.70 (s, 1.5H), 3.57-3.34 (m, 1H), 3.08-2.88 (m, 1H), 2.78-2.58 (m, 2H), 2.47-2.34 (m, 1H). ¹³C NMR ε: 195.9, 164.4, 163.6, 159.3, 159.1, 158.8, 158.7, 143.5, 137.7, 137.4, 129.3, 129.1, 127.8, 120.7, 114.8, 114.6, 114.4, 59.3, 55.9, 44.2, 38.5, 38.2, 35.7, 35.5, 34.6, 34.5.

2-Amino-4-(4-methoxyphenyl)-7-(3,4-dimethoxyphenyl)-5-oxo-5,6,7,8-tetrahydro-4H-chromene-3-carbonitrile (1n). The product was prepared according to reference [2]. The product, 1n, was isolated as a pale yellow powder: mp =159.9-161.4° C. ¹H NMR ε: 7.23-6.69 (m, 7H), 4.37 (br t, 0.5H), 4.19 (br s, 0.5H), 3.72 (br s, 6H), 3.36 (s, 3H), 3.31-3.16 (m, 1H), 3.12-2.84 (m, 1H), 2.84-2.56 (m, 1H), 2.51 (br s, 2H), 2.84-2.26 (m, 1H). ¹³C NMR ε: 195.0, 195.0, 163.5, 162.6, 158.3, 158.2, 157.8, 157.7, 148.5, 147.42, 147.39, 136.7, 136.5, 135.1, 135.0, 128.2, 119.7, 118.6, 118.3, 113.8, 113.6, 113.5, 111.7, 111.5, 111.0, 110.7, 58.4, 58.3, 56.0, 55.5, 55.4, 55.36, 55.04, 55.00, 43.7, 37.5, 37.1, 34.8, 34.7, 34.1, 34.0.

2-Amino-4-(4-methoxyphenyl)-7-(naphthalene-1-yl)-5-oxo-5,6,7,8-tetrahydro-4H-chromene-3-carbo-nitrile (1o, UCPH-101). 4-Methoxybenzaldehyde (0.102 mL, 0.839 mmol) was added to a mixture of 5-(naphthalen-l-yl)cyclohexane-1,3-dione (0.204, 0.854 mmol), and malononitrile (0.0593 g, 8.98 mmol) in 99.9% ethanol (1.5 mL). 4-Methylmorpholine (0.093 mL, 0.844 mmol) was added and the mixture was stirred for 10 min. after which it was left to stand at room temperature for 17 h followed by 4½ h at 5° C. The fluid above the precipitate was decanted and left to stand at room temperature for 20 h. The precipitated crystals was filtered off and washed with ethanol. Yield: 0.222 g (63%) white crystals. Mp: 194.0-197.2° C. NMR ε: 8.20 (d, 1H), 7.92 (d, 1H), 7.81 (d, 1H), 7.61-7.47 (m, 2H), 7.44-7.35 (m, 1H), 7.35-7.28 (m, 1H), 7.05 (d, 2H), 6.98 (br s, 2H), 6.82 (d, 2H), 4.47-4.33 (m, 1H), 4.22 (s, 1H), 3.72 (s, 3H), 3.14-2.87 (m, 2H), 2.83-2.58 (m, 2H). ¹³C-NMR ε: 195.8, 163.4, 159.0, 158.5, 138.9, 137.2, 134.2, 131.2, 129.5, 129.0, 128.0, 127.1, 126.4, 126.1, 124.1, 123.7, 120.6, 114.29, 114.26, 59.2, 55.8, 43.8, 35.8, 34.2, 33.7.

2-Amino-4-methyl-7-(naphthalene-1-yl)-5-oxo-5,6,7,8-tetrahydro-4H-chromene-3-carbo-nitrile (1w, UCPH-102). 5-(Naphthalen-1-yl)cyclohexane-1,3-dione (50 mg, 0.2 mmol), malononitrile (14 mg, 0.2 mmol) and acetaldehyde (35 μL, 0.6 mmol) was stirred in 3 mL absolute ethanol at rt. for 5 min. N-methylmorpholine (3 μL, 27 μmol) was added and the reaction mixture was stirred for 5 h. The reaction mixture was concentrated with silica. The crude/silica mixture was purified by column chromatography on silica gel using ethyl acetate/heptane (3:4) as eluent. This afforded the title compound (62 mg, 0.18 mmol, 90%) as colorless solid. R_{f}=0.30 and Mp 204-206° C. NMR (300 MHz, CDC1₃) ε (ppm) 8.01 (dd, J=13.5, 8.1 Hz, 1H), 7.91-7.86 (m, 1H), 7.78 (dd, J=8.1, 3.0 Hz, 1H), 7.59-7.24 (m, 4H), 4.46 (s, 2H), 4.33-4.09 (m, 1H), 3.48-3.39 (m, 1H), 2.96-2.69 (m, 4H), 1.35 (d, J=6.6 Hz, 2H), 1.26 (d, J=6.6 Hz, 1H). ¹³C NMR (75 MHz, CDCl₃) ε (ppm) 196.0, 162.8, 157.6, 137.4, 134.0, 130.8, 129.2, 127.9, 126.5, 125.9, 125.4, 122.8, 122.3, 118.8, 116.3, 43.6, 34.4, 33.4, 24.9, 22.9, 22.2.

### REFERENCES

1. Klokol, G. V., et al., Aliphatic aldehydes in the synthesis of condensed 4-alkyl(cycloalkyl)-2-amino-3-cyano-4H-pyrans. Khimiya Geterotsiklicheskikh Soedinenii, 1999(10): p. 1363-1366.

2. Jin, T. S., et al., A clean one-pot synthesis of tetrahydrobenzo[b]pyran derivatives catalyzed by hexadecyltrimethyl ammonium bromide in aqueous media. Synlett, 2004(5): p. 871-873.

3. Balalaie, S., et al., Diammonium hydrogen phosphate: An efficient and versatile catalyst for the one-pot synthesis of tetrahydrobenzo[b]pyran derivatives in aqueous media. Synthetic Communications, 2007. 37(7-9): p. 1097-1108.

4. Devi, I. and P. J. Bhuyan, Sodium bromide catalysed one-pot synthesis of tetrahydrobenzo[b]pyrans via a three-component cyclocondensation under microwave irradiation and solvent free conditions. Tetrahedron Letters, 2004. 45(47): p. 8625-8627.

### FIGURES AND TABLES

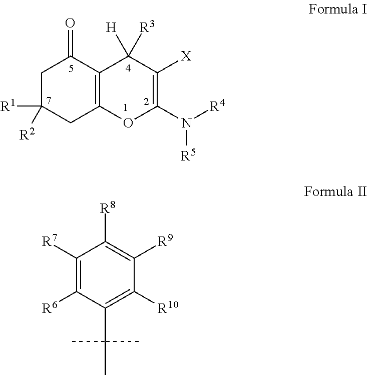

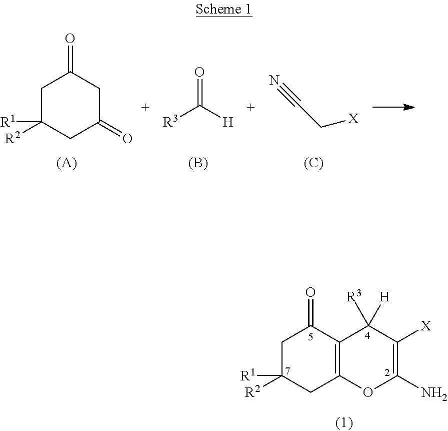

TABLE 1

| | | | | | R⁴ | | | |
|---|---|---|---|---|---|---|---|---|
| | R¹ | R² | R³ | X | R^{5′} | EAAT1 | EAAT2 | EAAT3 |
| 1a | 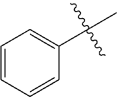 | H | 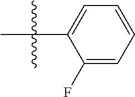 | CN | H, H | 4.7 [5.32 ± 0.03] | >100 [<4.0] | >100 [<4.0] |
| 1b | 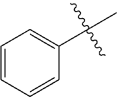 | H | 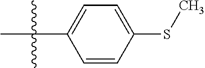 | CN | H, H | 2.7 [5.56 ± 0.05] | >100 [<4.0] | >100 [<4.0] |
| 1c | 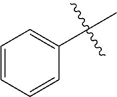 | H | 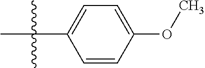 | CN | H, H | 2.3 [5.64 ± 0.05] | >100 [<4.0] | >100 [<4.0] |
| 1d | 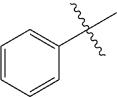 | H | 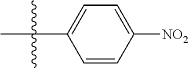 | CN | H, H | 3.6 [5.44 ± 0.04] | >100 [<4.0] | >100 [<4.0] |
| 1e | 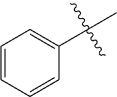 | H | 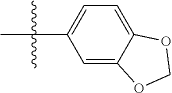 | CN | H, H | 4.0 [5.40 ± 0.05] | >300 [<3.5] | >300 [<3.5] |
| 1f | 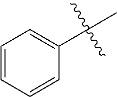 | H | 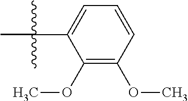 | CN | H, H | ~50 [~4.3] | >100 [<4.0] | >100 [<4.0] |
| 1g | 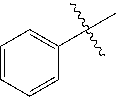 | H | 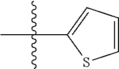 | CN | H, H | 14 [4.85 ± 0.04] | >100 [<4.0] | >100 [<4.0] |
| 1h | 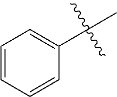 | H | 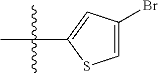 | CN | H, H | 4.7 [5.33 ± 0.04] | >100 [<4.0] | >100 [<4.0] |
| 1i | 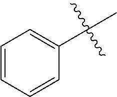 | H | 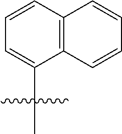 | CN | H, H | 7.4 [5.13 ± 0.05] | >100 [<4.0] | >100 [<4.0] |
| 1j | 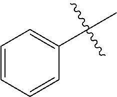 | H | 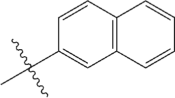 | CN | H, H | 3.6 [5.44 ± 0.04] | >100 [<4.0] | >100 [<4.0] |
| 1k | 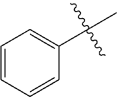 | H | 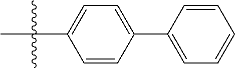 | CN | H, H | 2.1 [5.67 ± 0.04] | >100 [<4.0] | >100 [<4.0] |
| 1l | 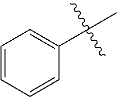 | H | 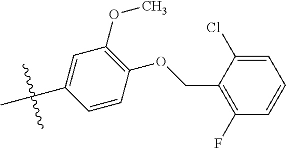 | CN | H, H | 4.3 [5.37 ± 0.03] | >100 [<4.0] | >100 [<4.0] |
| 1m | 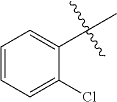 | H | 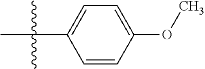 | CN | H, H | 3.2 [5.49 ± 0.04] | >300 [<3.5] | >300 [<3.5] |
| 1n | 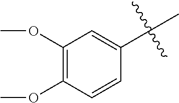 | H | 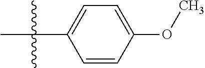 | CN | H, H | 11 [4.97 ± 0.05] | >300 [<3.5] | >300 [<3.5] |
| 1o, UCPH-101 | 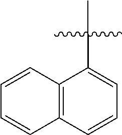 | H | 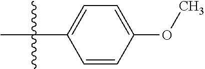 | CN | H, H | 0.66 [6.18 ± 0.08] | >300 [<3.5] | >300 [<3.5] |
| 1p | 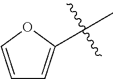 | H | 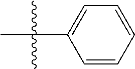 | CN | H, H | 7.3 [5.13 ± 0.03] | >300 [<3.5] | >300 [<3.5] |
| 1q | 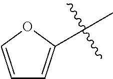 | H | 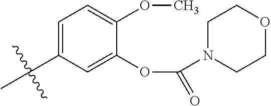 | CN | H, H | ~10 0 [~4.0] | >300 [<3.5] | >300 [<3.5] |
| 1r | 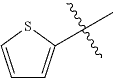 | H | 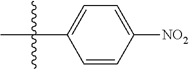 | CN | H, H | 9.9 [5.00 ± 0.04] | >300 [<3.5] | >300 [<3.5] |
| 1s | 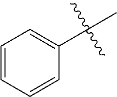 | H | 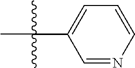 | CN | H, H | ~30 [~4.5] | >300 [<3.5] | >300 [<3.5] |
| 1t | 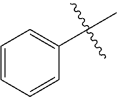 | H | 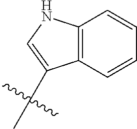 | CN | H, H | >100 [<4.0] | >100 [<4.0] | >100 [<4.0] |
| 1u | 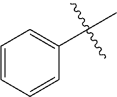 | H | 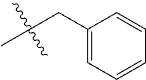 | CN | H, H | ~30 [~4.5] | >100 [<4.0] | >100 [<4.0] |
| 1v | 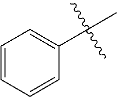 | H | Me | CN | H, H | 9.2 [5.04 ± 0.05] | >300 [<3.5] | >300 [<3.5] |
| 1w, UCPH- 102 | 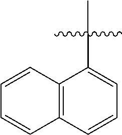 | H | Me | CN | H, H | 0.58 [6.25 ± 0.11] | >300 [<3.5] | >300 [<3.5] |

1. The use of a chemical substance which chemical structure is described by Formula I as inhibitors of EAAT1/GLAST, wherein:
R¹ is aryl, fused aryl, a carbocyclic group, a heterocyclic group, a heteroaryl group, alkenyl, alkynyl, arylalkyl, arylalkenyl, arylalkynyl, heteroarylalkyl, heteroarylalkenyl, heteroarylalkynyl, cycloalkylalkyl, heterocycloalkyl; or R¹ and R² are taken together to form a carbocycle or heterocycle; and
R² is hydrogen, C₁₋₁₀ alkyl, haloalkyl, aryl, fused aryl, a carbocyclic group, a heterocyclic group, a heteroaryl group, alkenyl, alkynyl, arylalkyl, arylalkenyl, arylalkynyl, heteroarylalkyl, heteroarylalkenyl, heteroarylalkynyl, cycloalkylalkyl, heterocycloalkyl, hydroxyalkyl, amino alkyl or thioalkyl; and
R³ is C₁₋₁₀ alkyl, haloalkyl, aryl, fused aryl, a carbocyclic group, a heterocyclic group, a heteroaryl group, alkenyl, alkynyl, arylalkyl, arylalkenyl, arylalkynyl, heteroarylalkyl, heteroarylalkenyl, heteroarylalkynyl, cycloalkylalkyl, heterocycloalkyl, hydroxyalkyl, amino alkyl or thioalkyl; and
R⁴ and R⁵ are independently hydrogen, C₁₋₁₀ alkyl, haloalkyl, aryl, fused aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl or aminoalkyl; or R⁴ and R⁵ are taken together to form a heterocycle; and
X is halogen, alkyl, aryl, heteroaryl, —NO₂ or —CN.

2. The use of a chemical substance which chemical structure is covered by claim 1 as inhibitors of EAAT1/GLAST, wherein:
R¹⁻⁵ are independently a phenyl ring with R⁶⁻¹⁰ (Formula II) independently: hydrogen, C₁₋₁₀ alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, nitro, amino, cyano, acylamino, hydroxy, thiol, acyloxy, azido, alkoxy, carboxy, carbonylamido, alkylthiol, halogen, haloalkyl, aryl, aryloxy, arylthioxy fused aryl, a carbocyclic group, a heterocyclic group, a heteroaryl group, C₁₋₁₀ alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, nitro, amino, cyano, acylamido, hydroxy, thiol, acyloxy, azido, alkoxy, carboxy, carbonylamido or alkylthiol; or
R⁶ and R⁷, or R⁷ and R⁸, or R⁸ and R⁹, or R⁹ and R¹⁰, are taken together to form a carbocycle or heterocycle, including —OCH₂O—, —OCF₂O—, —(CH₂)₃—, —(CH₂)_{4—, —OCH}₂CH₂O—, —CH₂N(R)CH₂—, —CH₂CH₂N(R)CH₂—, —CH₂N(R)CH₂CH₂—, —CH═CH═CH═CH—, and —N=CH—CH=N—.

3. The chemical structures described by Formula I, wherein R¹ is naphthyl, R^{2,4,5} are hydrogen and R³ is C₁₋₁₀ alkyl, haloalkyl, aryl, fused aryl, a carbocyclic group, a heterocyclic group, a heteroaryl group, alkenyl, alkynyl, arylalkyl, arylalkenyl, arylalkynyl, heteroarylalkyl, heteroarylalkenyl, heteroarylalkynyl, cycloalkylalkyl, heterocycloalkyl, hydroxyalkyl, amino alkyl or thioalkyl.

4. The chemical structures covered by claim 3 wherein the following substituents are present independently on the naphthyl group: C₁₋₁₀ alkyl, halo, hydroxyl, hydroxylalkyl, amino, alkyloxy, phosphorous, thioalkyl.

5. The use of a substance covered by claim 1-4 for the in-vitro or in-vivo characterization or studying of the EAAT1 subtype, such as its function, distribution in the central nervous system, and in studies of the physiological and pathological functions of the EAAT1/GLAST subtype in native tissues, cultured neurons, and/or animal models for CNS disorders.
